(19) United States
(12) Patent Application Publication
(10) Pub. No.: US 2019/0231926 A1
(43) Pub. Date: Aug. 1, 2019

(54) BONE GRAFT COMPOSITION WITH OSTEOGENIC CAPACITY

(71) Applicant: MAXIGEN BIOTECH INC., TAOYUAN CITY (TW)

(72) Inventors: SUNG-CHING CHEN, NEW TAIPEI CITY (TW); PO-HONG LAI, NEW TAIPEI CITY (TW); KAI-CHUN CHANG, TAOYUAN CITY (TW); YU-CHEN HSU, TAICHUNG CITY (TW)

(21) Appl. No.: 16/260,810

(22) Filed: Jan. 29, 2019

Related U.S. Application Data
(60) Provisional application No. 62/624,055, filed on Jan. 30, 2018.

(51) Int. Cl. A61L 27/12; A61K 38/18; A61L 27/24; A61L 27/36; A61L 27/56

(52) U.S. Cl. CPC A61L 27/12; A61K 38/1875; A61L 27/24; A61F 2/28; A61L 27/56; A61L 2430/02; A61L 27/365

(57) ABSTRACT

A bone graft composition promoting osteogenic capacity is provided. The bone graft composition includes an osteoinductive component including statins and a biodegradable polymer and an osteoconductive matrix including a biodegradable calcium phosphate ceramic, wherein the amount of the calcium phosphate ceramic is about 70 to 95 wt % based on the total weight of the bone graft composition. The bone graft composition can achieve the optimal release control characteristics of the statins in the osteoinductive component.

### CROSS-REFFERENCE TO RELATED APPLICATION

This application claims priority to and the benefit of, pursuant to 35 U.S.C. § 119, U.S. provisional patent application Ser. No. 62/624,055 filed on Jan. 30, 2018, the disclosure of which is incorporated herein in its entirety by reference.

### BACKGROUND OF THE INVENTION

### 1. Field of the Invention

The present invention relates to a bone graft applied to bone healing and regeneration, more particularly to a bone graft that promotes the osteoinductive ability including an osteoconductive scaffold and an osteoinductive component.

### 2. Description of the Related Art

A variety of synthetic bone graft substitutes have been used clinically in order to avoid the complications due to using autograft and the risks which allograft and xenograft may bring, such as disease transmittance and immunologic response, and the like. Since natural human bone mainly includes calcium phosphate as inorganic part and collagen as organic part, both biocompatible materials are commonly used as biodegradable bone graft scaffold. Great biocompatibility and porous structure provide cells with a suitable habitat to grow. Ideally, a bone tissue starts to regenerate as long as the scaffold degrades, and the scaffold which fills bone defect sites can be completely replaced by a newly grown bone. Nevertheless, due to lack of osteoinductivity among most synthetic bone grafts, nonunion, delay, or failure of bone healing commonly occurs clinically, leading to a great number of risks. Hence, in order to reduce risks and improve the outcome of the bone graft surgery, several strategies have been taken to increase the osteogenic capacity of synthetic bone grafts.

Statins, known as 3-hydroxy-2-methyl-glutaryl coenzyme A (HMG-CoA) reductase inhibitors, have been widely used to lower cholesterol and provide treatments for hyperlipidemia and arteriosclerosis. Statins can be also used to modulate bone formation, BMP mRNA expression, inflammation, and angiogenesis.

Simvastatin, mevastatin, and lovastatin were firstly shown to stimulate bone formation in vivo in 1999. Statins have thereafter been regarded as a potential alternative of recombinant human growth factors to provide osteoinductivity. Clinically, since rhBMP has a great capability of bone restoration, the rhBMP has thus been applied to bone surgery. However, adverse events (complications) frequently occur together with it. For instance, although a combination product of collagen matrix and rhBMP-2, Infuse ® Bone Graft launched by Medtronic Inc., has provided both osteoinductivity and osteoconductivity, there has been some reports on increasing risks of severe dose-dependent complications and adverse events, such as Ectopic Bone Formation, Vertebral Osteolysis and Subsidence, Postoperative Radiculitis, severe Hematoma, Seroma, and the like. These might be due to the instability of growth factors, an inevitable high dose of clinical products, and a lack of controlled release ability.

Over the past decades, numerous studies have been conducted on the usability of statins (small, stable, and relatively safe molecules) on bone regeneration clinically and concluded that local delivery with controlled release of statin is crucial to the enhancement of bone regeneration and the decrease of the unwanted inflammation near the implanted site. Hence, there is a growing need to develop a local delivery system of statins combined with the osteoconductive scaffold in an attempt to promote the feasibility of statins used as bioactive molecules to induce bones to grow.

### SUMMARY OF THE INVENTION

The present invention relates to a bone graft composition which promotes the osteoinductive ability and the osteogenic capacity. Certain embodiments of the present invention include, but are not limited to, a bone graft composition including a collagen matrix, calcium phosphate, or a combination thereof, and an osteoinductive molecule. Wherein the collagen matrix, the calcium phosphate, and the combination thereof form an osteoconductive component. The addition of osteoinductive molecules enhances the osteoinductive ability of the bone graft.

The present invention aims to provide a bone graft composition including an osteoinductive component containing statins and a biodegradable polymer and an osteoconductive matrix containing a biodegradable calcium phosphate ceramic. The calcium phosphate ceramic accounts for approximately 70 to 95 wt % of the total weight of the bone graft composition.

Preferably, the bone graft composition further includes collagen and the collagen accounts for approximately 5 to 20 wt % of the total weight of the bone graft composition.

Preferably, the statins have a drug concentration of approximately 0.3 to 0.7 mg/cc in the bone graft composition.

Preferably, the statins are selected from a group consisting of simvastatin, mevastatin, lovastatin, atorvastatin, pravastatin, fluvastatin, cerivastatin, rosuvastatin, pitavastatin, velostatin, eptastatin, and fluindostatin.

Preferably, the biodegradable polymer includes polylactic acid (PLA), polyglycolic acid (PGA), polylactic acid-glycolic acid copolymer (PLGA), polycaprolactone (PCL), or polyanhydrides.

Preferably, the calcium phosphate ceramic is selected from a group consisting of tricalciumphosphate (TCP), hydroxyapatite (HAP), dicalcium phosphate dehydrate (DCPD), and any combination thereof

Preferably, the calcium phosphate ceramic is a dicalcium phosphate dehydrate (DCPD)/hydroxyapatite (HAP) complex and a molar ratio of dicalcium phosphate dehydrate and hydroxyapatite ranges from 80:20 to 60:40.

Preferably, the calcium phosphate ceramic is a hydroxyapatite (HAP)/tricalciumphosphate (TCP) complex and a molar ratio of hydroxyapatite and tricalciumphosphate ranges from 90:10 to 50:50.

The bone graft composition of the present invention has the following advantages:

(1) The bone graft composition of the present invention may be widely applied to the manufacture of biomedical materials for various biomedical purposes. In particular, the bone graft composition having osteoconductivity and osteoinductivitymay be used as a bone substitute which induces bones to grow rapidly.

(2) The statins in the bone graft composition of the present invention may be continuously released for up to at least three weeks to stimulate cells to secrete BMP-2 so as to induce the formation of bones. In the meantime, because of the drug controlled release ability, the occurrence of adverse events such as dose-dependent complications can be reduced.

### BRIEF DESCRIPTION OF THE DRAWINGS

FIG. 1 depicts a flow chart of a manufacturing method of the bone graft composition according to one embodiment of the present invention.

FIG. 2 depicts a flow chart of a manufacturing method of the bone graft composition according to another embodiment of the present invention.

FIG. 3 depicts a graph showing the drug release accumulation test of Example 1 of the present invention, Comparative example 1, and Comparative example 2.

FIG. 4 depicts a graph showing the drug release accumulation test of Example 1 to Example 5 of the present invention and Comparative Example 2, wherein (a) depicts a drug concentration calibration curve, and (b) depicts a graph of the drug release accumulation test result.

FIG. 5 depicts a graph showing the cytotoxicity test of Example 1 of the present invention, Comparative example 1, and Comparative Example 2, wherein (a) depicts a result of 24-hour cell culture, and (b) depicts a result of 48-hour cell culture.

FIG. 6 depicts a tissue slice diagram and a quantitative graph showing the bone regeneration test of Example 1 of the present invention, Comparative Example 1, and Comparative Example 2, wherein (a) depicts a tissue slice diagram of the bone regeneration test of Comparative Example 1, (b) depicts a tissue slice diagram of the bone regeneration test of Comparative Example 2, (c) depicts a tissue slice diagram of the bone regeneration test of Example 1, and (d) depicts a quantitative graph of bone regeneration.

FIG. 7 depicts a tissue slice diagram and a quantitative graph showing the bone regeneration test of Example 6 and Example 7 of the present invention and Comparative Example 3, wherein (a) depicts a tissue slice diagram of the bone regeneration test of Comparative Example 3, (b) depicts a tissue slice diagram of the bone regeneration test of Example 6, (c) depicts a tissue slice diagram of the bone regeneration test of Example 7, and (d) depicts a quantitative graph of bone regeneration.

### DESCRIPTION OF THE PREFERRED EMBODIMENTS

The present invention is to be further described in detail by the following preferred embodiments together with the drawings. It should be noted that the experimental data disclosed in the following embodiments are used to explain the technical features of the present invention, and are not intended to limit the patterns to be implemented.

Definitions

The term “approximately” as used herein refers to values included in a range, such as errors in material proportion, errors in drug concentration values, or the difference between subjects in an experiment. The term typically refers to variability of values equal to approximately or less than 1%, 2%, 3%, 4%, 5%, 6%, 7%, 8%, 9%, 10%, 11%, 12%, 13%, 14%, 15%, 16%, 17%, 18%, 19%, or 20% as the case may be.

The term “effective dose” is an outcome expected to occur in medical science, such as a daily released dose of a drug in bone graft composition defined in the present invention. Medically expected results can be objective (That is, results based on some tests or measurements) or subjective (That is, results based on indication or effectiveness given by subjects).

The term “statins” as used herein is the drug currently used for the treatment of hyperlipidemia, which is considered to be a highly safe pharmaceutical composition. Statins as used herein include, but are not limited to, simvastatin, mevastatin, lovastatin, atorvastatin, pravastatin, fluvastatin, cerivastatin, rosuvastatin, pitavastatin, velostatin, eptastatin, or fluorostatin. Owing to similar mechanism among statins, simvastatin is taken as examples in the following embodiments. However, the present invention is not limited herein. For instance, lovastatin may also be used in the bone graft composition of the present invention.

The term “biodegradable” as used herein refers to an expected biocompatible polymer which may be biodegradable. That is, the polymer can be chemically and/or biologically degraded in a physiological environment (in the body). The “biodegradable” polymer as used herein, which has no significant toxic effects on cells, may be reused or discarded by cell mechanism (biodegradable) and/or by chemical process (such as hydrolysis) (chemically degradable) when introduced into cells. In one embodiment, the biodegradable polymer and the degradation byproducts thereof may be biocompatible.

The term “calcium phosphate ceramic” as used herein refers to a synthetic bone substitute material including calcium phosphate as a main component. Calcium phosphate-based materials suitable for use are known in the art including, but not limited to, tricalciumphosphate (TCP), hydroxyapatite (HAP), dicalcium phosphate dehydrate (DCPD), or any combination thereof. In a preferred embodiment, the calcium phosphate ceramic of the present invention has a calcium-to-phosphorus (Ca/P) ratio comparable to natural bone minerals. In another preferred embodiment, the calcium phosphate ceramic is manufactured by freezing and drying dicalcium phosphate dehydrate (DCPD)/hydroxyapatite (HAP) complex or hydroxyapatite (HAP)/tricalciumphosphate (TCP) complex.

The term “collagen” as used herein is used to denote an extracellular family of fibrous proteins characterized by a stiff triple helix structure. The triplex collagen peptide chains “a chains” are intertwined with each other to form helical molecules. In addition, the “collagen” in the present invention contains different types of collagen and includes natural collagen or recombinant collagen, which can be used for preparing the bone graft composition of the present invention. In a preferred embodiment, the collagen of the present invention may be Type I collagen, which may be extracted from animal tissues, such as cattle, pigs, horses, and the like. However, the present invention is not limited thereto.

### Embodiment

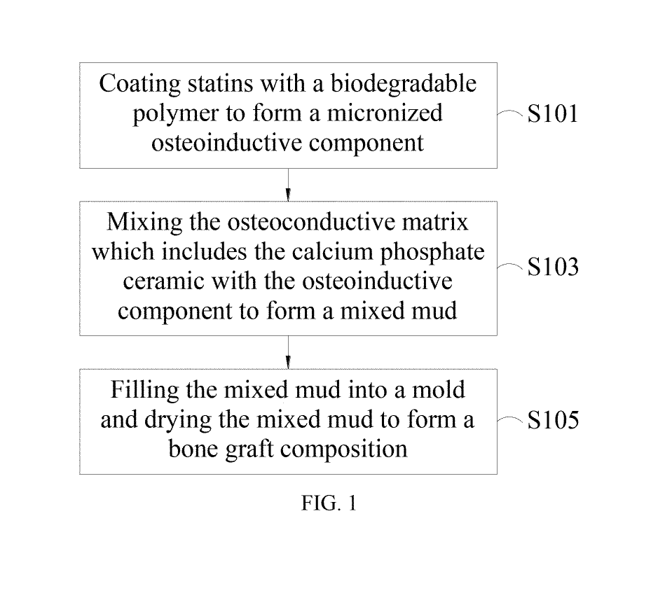

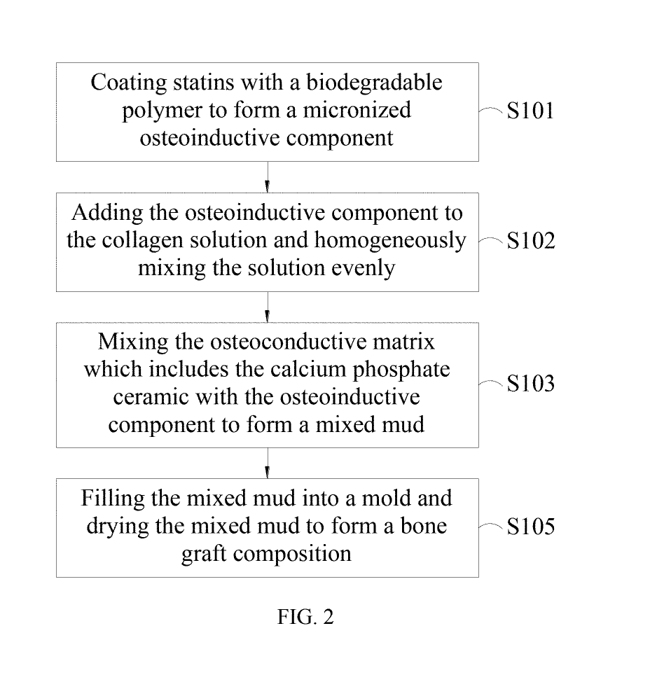

Please refer to FIG. 1 and FIG. 2, which respectively depict a flow chart of a manufacturing method of the bone graft composition according to one embodiment and another embodiment of the present invention. Please refer to FIG. 1 first, the present invention according to one embodiment provides a manufacturing method of the bone graft composition, including the following steps: (S101) coating statins with a biodegradable polymer to form a micronized osteoinductive component, wherein the osteoinductive component may have a particle size of 5 to 250 μm; (S103) mixing the osteoconductive matrix which includes the calcium phosphate ceramic with the osteoinductive component to form a mixed mud; and (S105) filling the mixed mud into a mold with a desired shape and drying or freeze-drying the mixed mud to form a bone graft composition having a drug stable release. In addition, according to the desired shape, a bone graft composition with a specific specification may be obtained by cutting, for example, a cubic particle shape, a sheet shape, or a block shape.

Please refer to FIG. 2. According to another embodiment, collagen may be used in the bone graft composition to stimulate the biological structure of natural human extracellular tissue and maintain as well as disperse calcium phosphate particles and osteoinductive components Therefore, the manufacturing method may further include: (S102) adding the osteoinductive component to a collagen solution and homogeneously mixing the solution evenly before proceeding to (S103), wherein the collagen solution may be prepared by extracting and purifying animal tissues to obtain a collagen mud, and then formulating with an alcohol solution, such as isopropanol. Preferably, the collagen is Type I collagen, and the total weight of the collagen in the bone graft composition may be approximately 5 to 20 wt %, preferably approximately 8 to 15 wt %, and most preferably approximately 10 to 12 wt %. In a particular embodiment, the collagen may account for 6 wt %, 7 wt %, 8 wt %, 9 wt %, 10 wt %, 11 wt %, 12 wt %, 13 wt %, 14 tt %, 15 wt %, 16 wt %, 17 wt %, 18 wt %, 19 wt %, or 20 wt % of the total weight of the bone graft composition.

In an embodiment of the present invention, the calcium phosphate ceramic used in the bone graft composition may include tricalciumphosphate (TCP), hydroxyapatite (HAP), dicalcium phosphate dehydrate (DCPD), or biphasic calcium phosphate. For instance, the HAP/TCP complex or the HAP/DCPD complex provides great biocompatibility and osteoinductivity, and is commonly used as synthetic bone grafts in the art. In some embodiments, the ratio of the calcium phosphate ceramic to the bone graft composition may be from approximately 70 to 95 wt %, preferably from approximately 75 to 90 wt %, most preferably from approximately 80 to 90 wt %. In a particular embodiment, the ratio of the calcium phosphate ceramic to the bone graft composition may be 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, or 89 wt %. Furthermore, in order to be well mixed with collagen, the calcium phosphate ceramic may have a particle size of 0.5 to 3 mm in a calcium phosphate irregular shape, preferably 0.1 mm to 3 mm, more preferably 0.5 mm to 1 mm. In another embodiment, when a hydroxyapatite (HAP)/tricalciumphosphate (TCP) complex is used, the molar ratio of HAP to TCP may be from approximately 90:10 to approximately 50:50, preferably from approximately 70:30 to approximately 60:40, such as approximately 90:10, approximately 80:20, approximately 70:30, approximately 60:40, approximately 50:50, or approximately 40:60, most preferably 60:40. In yet another embodiment, when a dicalcium phosphate dehydrate (DCPD)/hydroxyapatite (HAP) complex is used, the molar ratio of DCPD to HAP may range from approximately 80:20 to approximately 60:40, preferably from approximately 70:30 to approximately 60:40, such as approximately 90:10, approximately 80:20, approximately 70:30, approximately 60:40, or approximately 50:50, and most preferably 70:30.

In an embodiment of the present invention, the statins are preferably small-molecule statins rather than large and unstable growth factors. The statins may be selected from the group consisting of simvastatin, mavastatin, lovastatin, atorvastatin, and the like. In some embodiments, the statin molecules may be combined with a biodegradable polymer used as a pharmaceutical carrier, such as polymer microparticles formed by, for example, Polylactic acid (PLA), polyglycolic acid (PGA), polylactic acid-glycolic acid copolymer (PLGA), polycaprolactone (PCL), or polyanhydrides. The biodegradable polymer allows statins to remain in the matrix and is released at a controlled rate to avoid the great burst release of drugs.

According to previous studies, a certain dose control mechanism is extremely important. According to an embodiment of the present invention, the drug concentration of the statins in the bone graft composition may be controlled in a range from approximately 0.3 to 0.7 mg/cc, preferably approximately 0.4 to 0.6 mg/cc. In addition, the ideal therapeutic concentration of statins released daily to induce bone to grow should be no more than 10 04, preferably 0.01 μM to 5 μM, more preferably 0.01 04 to 2 μM. In one embodiment, the drug concentration of the statins in the bone graft composition may be 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, or 0.7 mg/cc. In the present invention, the inherent viscosity of the biodegradable polymer can be 0.55 dL/g. the combination of the biodegradable polymer and the osteoconductive scaffold provides superior controlled release capacity, ensuring that the released drug concentration may be sustainably satisfied the desired range. By constantly maintaining this therapeutic concentration of the statins for a period of time, for example at least about 21 days, the drug may be sustainably and steadily released during the most critical period of bone regeneration, thereby improving the performance of bone growth and bone healing in the bone defect site and avoiding possible side effects triggered by statin molecules.

In a preferred embodiment, the biodegradable polymer may be a polylactic acid-glycolic acid copolymer (PLGA) which is copolymerized from lactic acid and glycolic acid and has biocompatibility and biodegradability. The lactic acid may be L-lactic acid, D-lactic acid, or D, L-lactic acid. The degradation rate of PLGA may be adjusted by changing the ratio of lactic acid to glycolic acid. In some embodiments, the monomer ratio of lactic acid to glycolic acid in the PLGA may be from 1:4 to 4:1, such as approximately 1:3, approximately 2:3, approximately 1:1, approximately 3:2, or approximately 3:1, the most preferably 1:1. In a particular embodiment, the monomer ratio of lactic acid to glycolic acid may be 4:1, 3:1, 7:3, 13:7, 3:2, 11:9, 1:1, 9:11, 2:3, 7:13, 3:7, 1:3, or 1:4.

The bone graft composition prepared according to the aforementioned method of the present invention may be delivered to a bone defect site requiring bone tissue regeneration, which may be caused by trauma (including open and closed fractures), diseases, congenital defects, or surgery, namely spinal repair (e.g., spinal fusion), long bone defect, cranial defect, iliac crest back filling, in the repair of tibial plateau, periodontal bone defect, maxillofacial bone defect, and the like. When delivered to the aforementioned bone defect site, the bone graft composition of the present invention has a fine bone filling property and also provides osteoconductivity (facilitating cells to grow) and osteoinductivity (promoting cells to secret BMP-2), thereby acquiring excellent bone healing effects. In addition, the statins in the bone graft composition of the present invention may be sustainably and steadily released for at least three weeks so that the risk of drug dose dependency may not exist when the drug continues to stimulate cells to secret BMP-2.

### EXAMPLE 1

### Preparation of FG/PLGA/SIM

Type I collagen is extracted from animal tissues, and then the purified collagen is formulated into a collagen solution with an isopropanol solution. Afterward, 150 mg of micron-sized PLGA (the monomer ratio of lactic acid: glycolic acid is 1:1)/Simvastatin (SIM) is added to the collagen solution. The solution is stirred evenly with a homogenizer. The calcium phosphate ceramic HAP/TCP complex (molar ratio 60:40) is further added. The solution is stirred evenly with a homogenizer to obtain a mixed mud. After uniformly mixed, the mixed mud is filled into a mold, placed in a freeze dryer for drying, and then cut to obtain a bone graft composition (FG/PLGA/SIM), wherein the calcium phosphate ceramic HAP/TCP and Type I collagen are used as an osteoconductive matrix (FG), and PLGA/SIM is used as an osteoinductive component. In the bone graft composition of Example 1 (FG/PLGA/SIM), the weight ratio of the collagen solution to HAP/TCP calcium phosphate ceramic is approximately 1:4, and the content of micron-sized particles PLGA/SIM is approximately 1 wt % of the collagen solution.

Please refer to Table 1 below, Examples 2-5 are identical to Example 1 in the manufacturing process and compositions thereof except different drug concentrations of simvastatin are used.

### EXAMPLE 6

### Preparation of BC/PLGA/SIM in a Low Concentration Group

The calcium phosphate monobasic monohydrate (MCPM) is mixed with HAP powder and liquid for injection to form a calcium phosphate ceramic of a DCPD/HAP complex (molar ratio 70:30). After adding 6 mg of micron-sized particles PLGA/simvastatin (SIM), the mixture was uniformly stirred by a homogenizer to obtain a mixed mud. After uniformly mixed, the mixed mud is filled into a mold, dried and de-molded, and then cut to obtain a bone graft composition (BC/PLGA/SIM), wherein the calcium phosphate ceramic DCPD/HAP is used as an osteoconductive matrix (BC), and PLGA/SIM is used as an osteoinductive component. In the bone graft composition of Example 6, the weight of the DCPD/HAP calcium phosphate ceramic is 99 wt %, the weight of the micron-sized particle PLGA/SIM is 1 wt %, and the concentration of simvastatin in the bone graft composition is approximately 0.34 mg/cc.

### EXAMPLE 7

### Preparation of BC/PLGA/SIM in a High Concentration Group

The calcium phosphate monobasic monohydrate (MCPM) is mixed with HAP powder and liquid for injection to form a calcium phosphate ceramic of a DCPD/HAP complex. After adding 12 mg of micron-sized particles PLGA/simvastatin (SIM), the mixture was uniformly stirred by a homogenizer to obtain a mixed mud. After uniformly mixed, the mixed mud is filled into a mold, dried and de-molded, and then cut to obtain a bone graft composition (BC/PLGA/SIM), wherein the calcium phosphate ceramic DCPD/HAP is used as an osteoconductive matrix (BC), and PLGA/SIM is used as an osteoinductive component. In the bone graft composition of Example 7, the weight of the DCPD/HAP calcium phosphate ceramic is 99 wt %, the weight of the micron-sized particle PLGA/SIM is 1 wt %, and the concentration of simvastatin in the bone graft composition is approximately 0.68 mg/cc.

### COMPARATIVE EXAMPLE 1

### FG

A bone graft (FG) contains only calcium phosphate ceramic HAP/TCP complex without drugs and biodegradable polymers.

### COMPARATIVE EXAMPLE 2

### FG/SIM

A drug-containing bone graft (FG/SIM) contains uncoated simvastatin and calcium phosphate ceramic HAP/TCP complex without biodegradable polymers, wherein the drug content per unit identical to the amount as in Example 1.

### COMPARATIVE EXAMPLE 3

### BC

A bone graft (BC) contains only calcium phosphate ceramic DCPD/HAP without drugs and biodegradable polymers.

According to the Examples and Comparative Examples as described above, the component of the compositions are shown in Table 1:

TABLE 1

| | | The weight ratio | | |
|---|---|---|---|---|
| | | of the collagen | The monomer | |
| | The molar ratio of | solution:the | ratio of Lactic | Drug |
| | the calcium | calcium phosphate | acid:glycolic | concentration |
| | phosphate ceramic | ceramic | acid in PLGA | of simvastatin |
| Example 2 | HAP/TCP 60:40 | 1:4 | 1:1 | 0.2 mg/cc |
| Example 3 | HAP/TCP 60:40 | 1:4 | 1:1 | 0.3 mg/cc |
| Example 4 | HAP/TCP 60:40 | 1:4 | 1:1 | 0.7 mg/cc |
| Example 5 | HAP/TCP 60:40 | 1:4 | 1:1 | 0.8 mg/cc |
| Example 6 | DCPD/HAP 70:30 | — | 1:1 | 0.34 mg/cc |
| Example 7 | DCPD/HAP 70:30 | — | 1:1 | 0.68 mg/cc |
| Comparative | HAP/TCP 60:40 | — | — | — |
| Example 1 | | | | |
| Comparative | HAP/TCP 60:40 | — | — | Same as |
| Example 2 | | | | Example 1 |
| Comparative | DCPD/HAP 70:30 | — | — | — |
| Example 3 | | | | |

Experimental Analysis

Drug Release Analysis

The samples of 100 mg of the FG group (Comparative Example 1), the FG/SIM group (Comparative Example 2), and the FG/PLGA/SIM group (Example 1) are respectively placed in a test tube containing 6 ml aqueous solution, and are continuously shaken (80 rpm) by an oscillator to perform an in vitro release test. The test tubes are centrifuged at 4000 rpm, and 4 ml of supernatant are respectively taken and 4 ml of fresh PBS solution is added to each of the mother test tube, per day for 3 weeks. The drug release concentration of simvastatin is measured by using HPLC, and accumulation is performed to obtain the result of the release accumulation concentration.

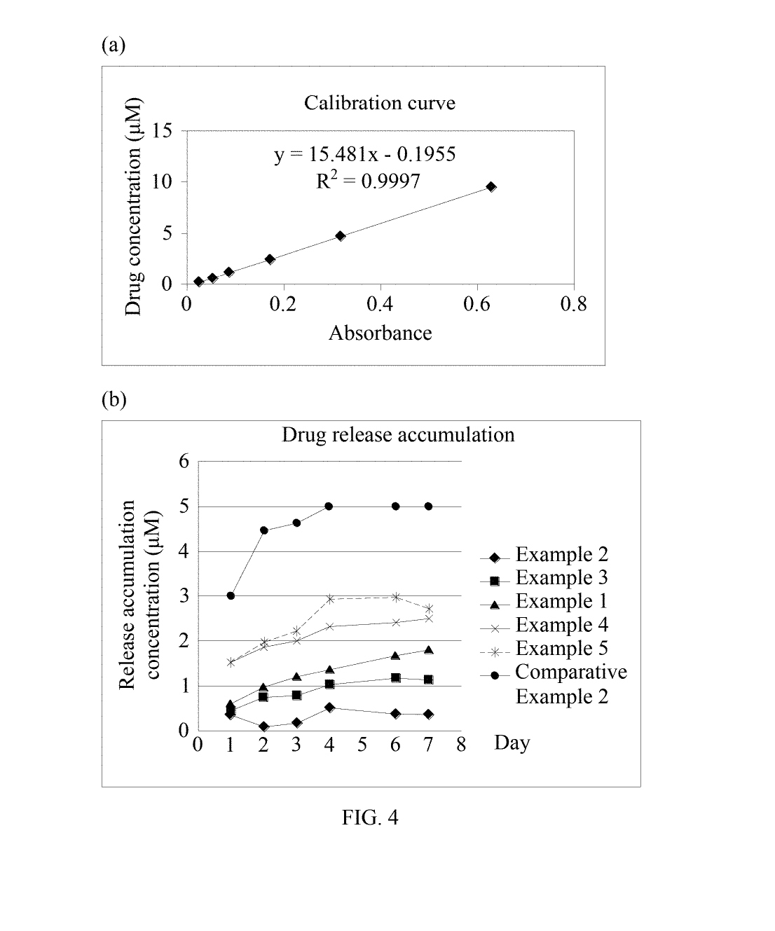

The samples of 100 mg of Examples 1 to 5 are respectively placed in a aqueous test tube containing 6 ml aqueous solution, and are continuously shaken (80 rpm) by an oscillator to perform an in vitro release test. The test tubes are centrifuged at 4000 rpm, and 4 ml of supernatant are respectively taken and 4 ml of fresh PBS solution is added to each of the mother test tube, per day for 7 days. The 238 nm wavelength absorption value of UV-Vis is detected at each time point every day, and the concentration is converted by referring to the calibration curve of FIG. 4(a) to obtain the result of the release accumulation concentration.

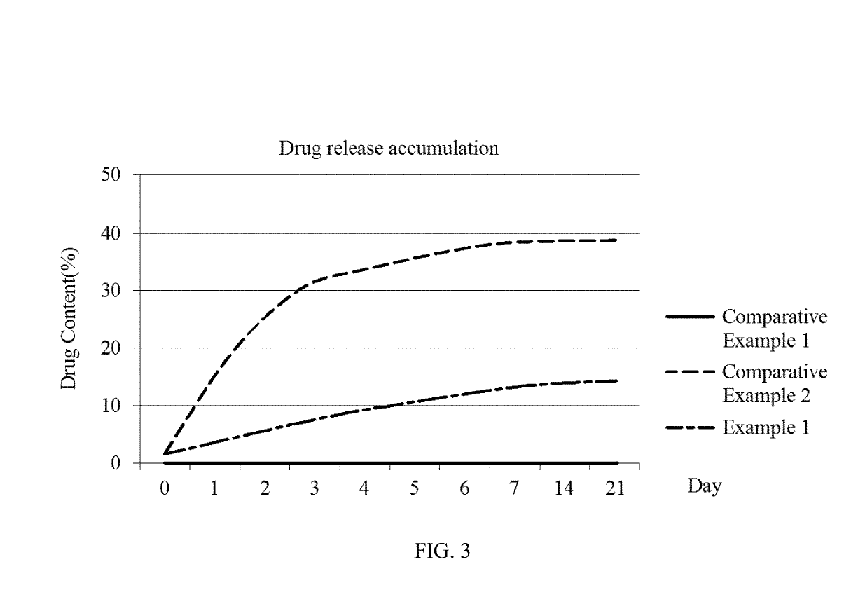

According to the result in FIG. 3, the curve slope of the drug release from day 1 to day 7 of the FG/SIM group is significantly higher than that of the FG/PLGA/SIM group, indicating that the drug is largely and explosively released in this period. However, no increasing tendency is found after 14 days. In contrast, the curve slope of the drug release in the FG/PLGA/SIM group is relatively steadily and slowly increased and can be released moderately for at least 3 weeks, indicating that the bone graft composition of the present invention does have a stable drug release with sustainable efficacy.

According to the result of FIG. 4(b), the drug release curve slopes of Example 1, Examples 3, and Example 4 all are steadily increased, and it is observed that the drug concentration released daily is approximately 0.01 μM to 2 μM. In contrast, although the drug concentration released daily in Examples 2 and Example 5 is not explosive compared with that in Comparative Example 2, it is found that the curves rise unsteadily, showing that both Example 2 and Example 5 are unable to be steadily released due to excessive or low drug contents. Accordingly, the experimental results indicate that the optimal drug concentration to which the present invention is applied ranges from 0.3 to 0.7 mg/cc.

Cytotoxicity Test (LDH Assay)

A cell compatibility test is performed with D1 cells. The supernatant is removed after the samples of 0.2 g/ml of the FG group (Comparative Example 1), the FG/SIM group (Comparative Example 2), and the FG/PLGA/SIM group (Example 1) are cultured with D1 cells in a 37 ° C. incubator for 72 hours. Next, cell culture is performed at a 96 well-plate and cell density of 10 cells/well for 24 or 48 hours. The supernatant of cell culture is taken. Then, LDH concentration is measured at O.D.490 nm using the LDH cytotoxicity detecting kit (Cat. No. 630117, CloneTech PT3947). This experiment is in accordance with ISO 10993-5 and conforms to ISO 10993-12.

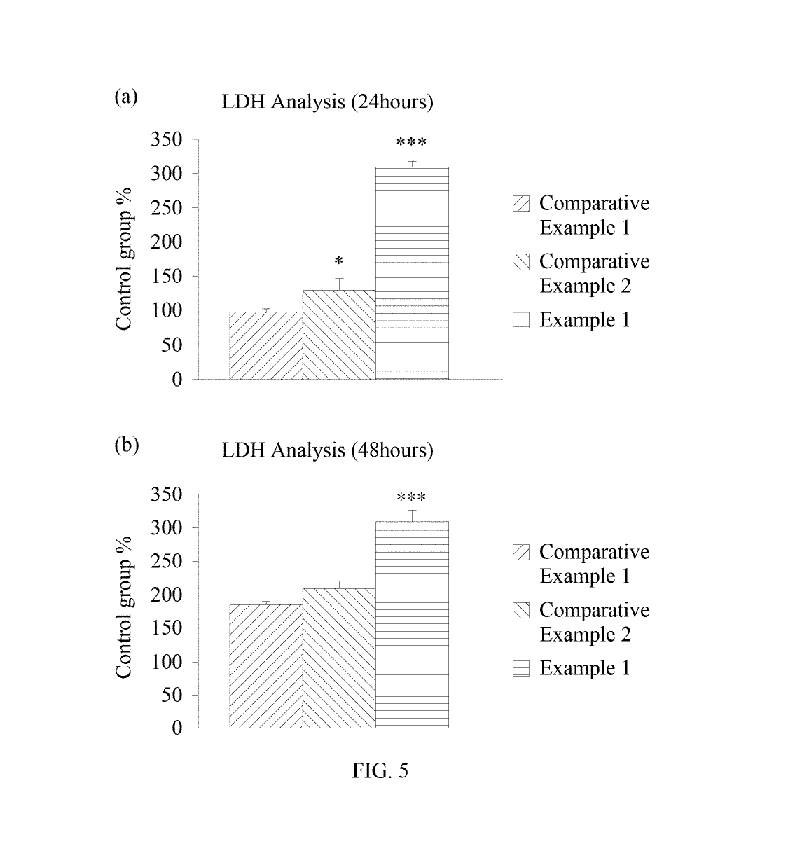

According to the results of FIG. 5 (a) and (b), it is shown that the cytotoxicity of the FG/SIM group is significantly higher than that of the FG group at either 24 hours or 48 hours, showing that statins produce greater toxic effects on cells without being coated with PLGA. In contrast, although the FG/PLGA/SIM group in FIG. 5(a) is only slightly higher than the FG group at 24 hours, there is no significant difference from the FG group after 48 hours in FIG. 5(b). This shows that the bone graft composition of the present invention does have excellent biocompatibility and is less harmful to cells.

Bone Regeneration Test

Rabbits are used as experimental subjects and randomly divided into (1) FG group (Comparative Example 1); (2) FG/SIM group (Comparative Example 2); (3) FG/PLGA/SIM group (Example 1), with each group consisting of four or five. The rabbits are fixed with the front facing the table after being anesthetized. Surgery is operated along the lateral intervertebral muscle space that reveals the regions of L4 and L5 transverse processes and the intertransverse processes thereof. After the cortex is removed from the transverse processes, the graft of Example 1, Comparative Example 1, and Comparative Example 2 are implanted. The muscles are properly placed for fixation, and suturing is done layer by layer. The animals are kept in cages and fed freely. The conditions after surgery are observed and recorded. X-ray is used for observation every two weeks after surgery. The rabbits are sacrificed on the twelfth week. The vertebrae L4 and L5 are taken and fixed with 10% of formalin for the uCT image analysis. Decalcification and tissue staining analysis are subsequently performed to observe whether new bone regeneration and surrounding tissues are inflamed. Bone growth status is assessed by a quantitative tissue staining method.

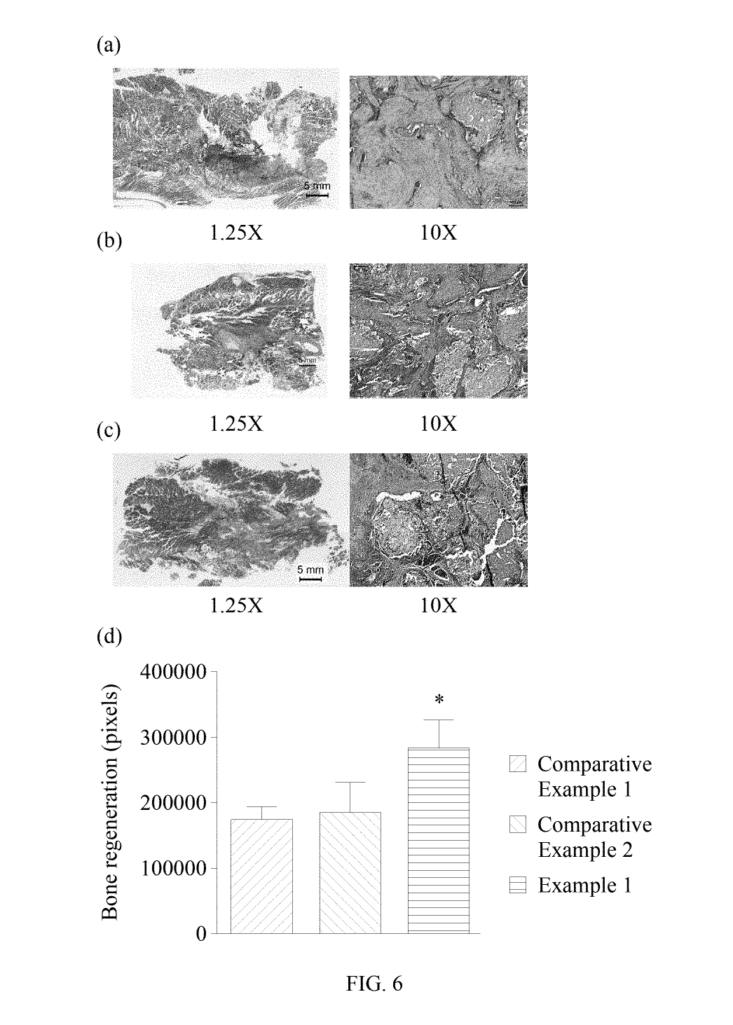

According to the results of tissue staining images in FIG. 6(a) to (c), the degree of bone regeneration of the FG/PLGA/SIM group (FIG. 6(c)) is indeed better than that of the FG group (FIG. 6(a)) and the FG/SIM group (FIG. 6(b)). Moreover, according to the quantitative results of FIG. 6(d), the FG/PLGA/SIM group is also significantly higher than the FG group and the FG/SIM group. Therefore, the bone graft composition of the present invention does achieve a great effect on inducing bones to grow rapidly.

In another experiment, New Zealand rabbits are used as experimental subjects and randomly divided into (1) BC group (Comparative Example 3); (2) BC/PLGA/SIM low concentration group (Example 6); (3) BC/PLGA/SIM high concentration group (Example 7), with each group consisting of four to five. Forelimb radius periosteums of the rabbits are removed after being anesthetized. One cm of bone is cut off from the center of the radius, and the bone grafts of each group are placed at the bone defect. After surgery, animals are kept in cages and fed freely. The conditions after surgery are observed and recorded. The rabbits are sacrificed on the sixth week to assess bone regeneration.

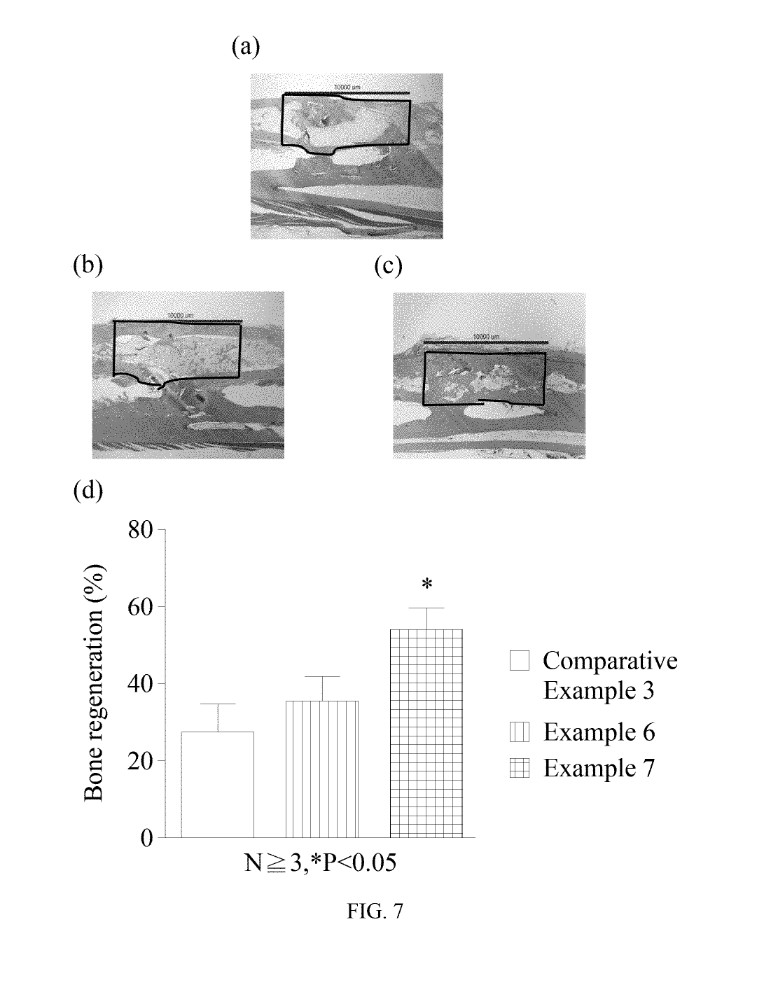

Based on the results of FIG. 7(a) to (d), although the difference between the BC/PLGA/SIM low concentration group (FIG. 7(b)) and the FG group (FIG. 7(a)) is not obvious, it can be seen that the bone regeneration effect of the BC/PLGA/SIM low concentration group is slightly better than that of the FG group according to the quantitative results of FIG. 7(d). The BC/PLGA/SIM high concentration group (FIG. 7(c)) is significantly superior to the FG group and the low concentration group in either images or quantitative results, indicating that the bone graft composition of the present invention may have the efficacy of promoting bone regeneration when containing statins with a drug concentration of approximately 0.3 to 0.7 mg/cc.

What is claimed is:

1. A bone graft composition, comprising:
an osteoinductive component comprising statins and a biodegradable polymer; and
an osteoconductive matrix comprising a biodegradable calcium phosphate ceramic, wherein the calcium phosphate ceramic accounts for approximately 70 to 95 wt % of a total weight of the bone graft composition.

2. The bone graft composition according to claim 1 further comprising collagen and the collagen accounts for approximately 5 to 20 wt % of the total weight of the bone graft composition.

3. The bone graft composition according to claim 1, wherein the statins have a drug concentration of approximately 0.3 to 0.7 mg/cc in the bone graft composition.

4. The bone graft composition according to claim 1, wherein the statins are selected from a group consisting of simvastatin, mevastatin, lovastatin, atorvastatin, pravastatin, fluvastatin, cerivastatin, rosuvastatin, pitavastatin, velostatin, eptastatin, and fluindostatin.

5. The bone graft composition according to claim 1, wherein the biodegradable polymer comprises Polylactic acid (PLA), polyglycolic acid (PGA), polylactic acid-glycolic acid copolymer (PLGA), polycaprolactone (PCL), or polyanhydrides.

6. The bone graft composition according to claim 1, wherein the calcium phosphate ceramic is selected from a group consisting of tricalciumphosphate (TCP), hydroxyapatite (HAP), dicalcium phosphate dehydrate (DCPD), and any combination thereof

7. The bone graft composition according to claim 6, wherein the calcium phosphate ceramic is a dicalcium phosphate dehydrate (DCPD)/hydroxyapatite (HAP) complex and a molar ratio of dicalcium phosphate dehydrate and hydroxyapatite ranges from 80:20 to 60:40.

8. The bone graft composition according to claim 6, wherein the calcium phosphate ceramic is a hydroxyapatite (HAP)/tricalciumphosphate (TCP) complex and a molar ratio of hydroxyapatite and tricalciumphosphate ranges from 90:10 to 50:50.
